(19) United States
(12) Patent Application Publication
(10) Pub. No.: US 2004/0230073 A1
(43) Pub. Date: Nov. 18, 2004

(54) Process for production of aspartame derivative, crystal thereof, novel production intermediate therefor, and process for production of intermediate thereof

(75) Inventors: Shigeru Kawahara, Kawasaki-shi (JP); Kazutaka Nagashima, Kawasaki-shi (JP); Kenichi Mori, Kawasaki-shi (JP); Tadashi Takemoto, Kawasaki-shi (JP); Eriko Ono, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 10/862,426

(22) Filed: Jun. 8, 2004

Related U.S. Application Data
(62) Division of application No. 10/117,205, filed on Apr. 8, 2002.
Application No. 10/117,205 is a continuation of application No. PCT/JP00/06933, filed on Oct. 4, 2000.

(30) Foreign Application Priority Data
Oct. 8, 1999 (JP) 11-288207
Oct. 8, 1999 (JP) 11-288208
Oct. 15, 1999 (JP) 11-294409
Nov. 18, 1999 (JP) 11-328099

(51) Int. Cl. C07K 5/04

(52) U.S. Cl. 560/40

(57) ABSTRACT

The present invention relates to a method of manufacturing aspartyl dipeptide ester compounds, which can be used as sweeteners.

### CROSS-REFERENCE TO RELATED APPLICATION

The present application is a continuation application of PCT/JP00/06933 filed Oct. 4, 2000, the entire contents of which are incorporated by reference.

### FIELD OF THE INVENTION

The present invention relates to processes for producing aspartyl dipeptide ester derivatives, such as N-[N-[3-(3-hydroxy-4-methoxyphenyl)-3-methylbutyl]-L-α-aspartyl]-L-phenylalanine 1-methyl ester, which are important sweeteners with a high degree of sweetness. This process may include processes for crystallization and employ novel aldehyde derivatives as intermediates.

### BACKGROUND OF THE INVENTION

In recent years, as eating habits have increased, excessive weight gain and obesity caused by excessive sugar intake has been more frequently observed. Additionally, diseases accompanied by such weight gain and obesity are becoming more prevalent. Accordingly, the development of a low-calorie sweetener (sweetening agent) that replaces sugar has been strongly in demand. Aspartame is widely used as a sugar substitute or sweetener; and is excellent in safety and sweetening quality. However, a drawback of aspartame is that is somewhat unstable.

The present inventors have found that N-[N-[3-(3-hydroxy-4-methoxyphenyl)-3-methylbutyl]-L-α-aspartyl]-L-phenylalanine 1-methyl ester, shown in the following formula, is useful as a sweetener and is excellent in stability. Furthermore, this ester compound has a degree of sweetness and therefore, has the advantage in cost.

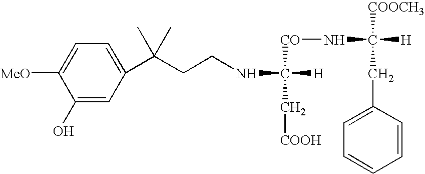

Because sweeteners are primarily used in foods and pharmaceuticals, which are to be consumed by a person, the sweeteners should be purified to high purity, that is essentially free of impurity and/or decomposed materials. Furthermore, where peptide-based sweeteners are employed, which may decompose rather easily, there exists a need to provide such sweeteners in a stable form, to prevent decomposition during storage and shipment.

In a process for producing N-[N-[3-(3-hydroxy-4-methoxyphenyl)-3-methylbutyl]-L-α-aspartyl]-L-phenylalanine 1-methyl ester a β-O-benzyl-α-L-aspartyl-L-phenylalanine methyl ester is reductively alkylated with 3-(3-benzyloxy-4-methoxyphenyl)-3-methylbutylaldehyde and NaB(OAc)₃H, which is followed by removing the benzyl moiety of a protecting group. However, 3-(3-benzyloxy-4-methoxyphenyl)-3-methylbutylaldehyde is prepared by a 7 step process starting from 3-hydroxy-4-methoxyacetophenone, as shown in the following reaction process 1, and therefore the compound is not well suited to be used to provide a industrially profitable process.

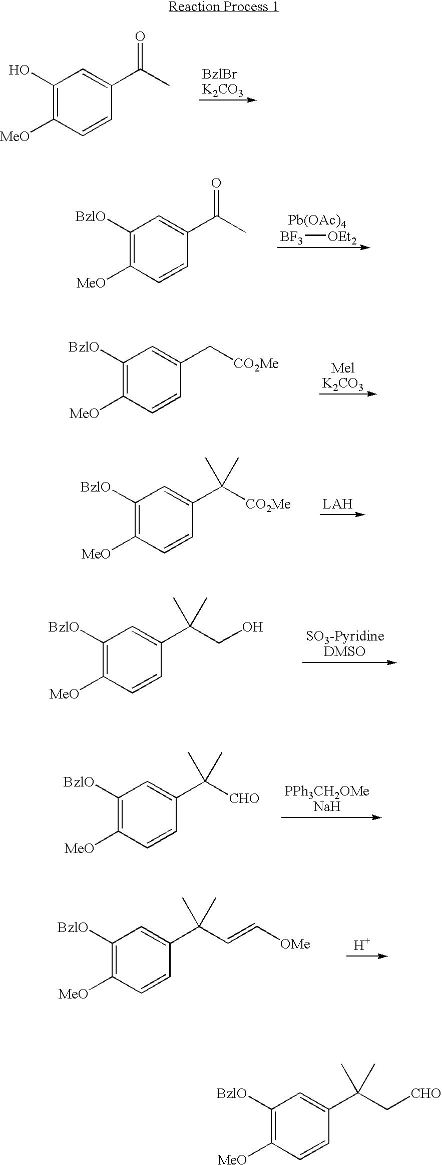

Therefore, a problem to be solved by the present invention is to provide an efficient process for producing N-[N-[3-(3-hydroxy-4-methoxyphenyl)-3-methylbutyl]-L-α-aspartyl]-L-phenylalanine 1-methyl ester. The invention also provides a practical and industrial process for purifying this ester compound and in particular for obtaining the ester compound in the crystalline form at a high purity.

### SUMMARY OF THE INVENTION

The present inventors have found that an aspartyl dipeptide ester derivative represented by formula (2) can be obtained in a process by reductively alkylating aspartame with an aldehyde represented by formula (1), preferably in the presence of catalyst, more preferably in the presence of hydrogen.

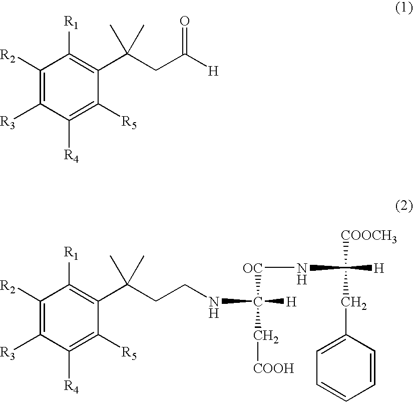

where in formulas (1) and (2), R₁, R₂, R₃, R₄ and R₅ are independently a hydrogen atom, a hydroxyl group, an alkoxy group having 1 to 3 carbon atoms, an alkyl group having 1 to 3 carbon atoms, a benzyloxy group and a hydroxyalkyloxy group having 2 or 3 carbon atoms, wherein two symbols of R₁ and R₂, or two symbols of R₂ and R₃ may form a methylene dioxy group, and

provided that in the formula (2), any one of R₁, R₂, R₃, R₄ and R₅ is not a benzyloxy group.

The inventors have also succeeded in synthesizing a novel 3-(3-hydroxy-4-methoxyphenyl)-3-methylbutylaldehyde, which can be used as an intermediate in the above production process

In one embodiment of the invention, the process comprises the steps outlined in the reaction process 2 depicted below.

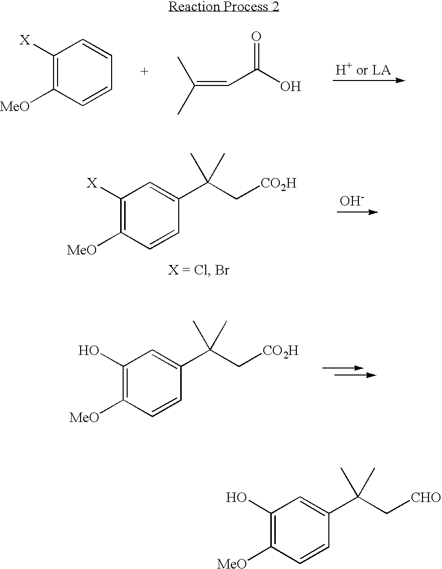

Another object of the present invention is 3-(3-hydroxy-4-methoxyphenyl)-3-methylbutylaldehyde, which can be employed as an intermediate for producing N-[N-[3-(3-hydroxy-4-methoxyphenyl)-3-methylbutyl]-L-α-aspartyl]-L-phenylalanine 1 -methyl ester and which is advantageous compared to a process for producing 3-(3-benzyloxy-4-methoxyphenyl)-3-methylbutylaldehyde described above.

The 3-(3-hydroxy-4-methoxyphenyl)-3-methylbutylaldehyde can be synthesized, for example, in a process where 2-halogenoanisole is reacted with 3-methylcrotonic acid, preferably in the presence of an acid. This reaction is followed by a conversion of a halogen atom in the 3-(3-halogeno-4-methoxyphenyl)-3-methylbutyric acid obtained into a hydroxyl group by alkaline hydrolysis, in the presence of a copper catalyst. The carboxylic acid is then converted to an aldehyde.

Another object of the invention is a process for producing N-[N-[3-(3-hydroxy-4-methoxyphenyl)-3-methylbutyl]-L-α-aspartyl]-L-phenylalanine 1-methyl ester at a high purity, which involves crystallization.

### BRIEF DESCRIPTION OF THE DRAWING

FIG. 1 is a powder X-ray diffraction pattern of N-[N-[3-(3-hydroxy-4-methoxyphenyl)-3-methylbutyl]-L-α-aspartyl]-L-phenylalanine 1-methyl ester in the crystalline form obtained in the present invention.

### DETAILED DESCRIPTION OF THE INVENTION

The present invention provides processes for producing aspartyl dipeptide ester derivative represented by formula (2) by reductively alkylating aspartame with the aldehyde represented by formula (1):

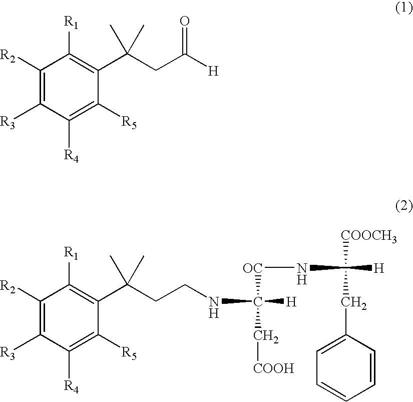

where in (1) and (2), R₁, R₂, R₃, R₄ and R₅ are independently a hydrogen atom, a hydroxyl group, an alkoxy group having 1 to 3 carbon atoms, an alkyl group having 1 to 3 carbon atoms, a benzyloxy group and a hydroxyalkyloxy group having 2 or 3 carbon atoms, wherein R₁ and R₂, or R₂ and R₃ may form a methylene dioxy group, and provided that in formula (2), any one of R₁, R₂, R₃, R₄ and R₅ is not a benzyloxy group.

In one embodiment, R₃ is a methoxy group, and R₁, R₂, R₄ and R₅ are hydrogen atoms. In another embodiment, R₃ is a hydroxyl group, and R₁, R₂, R₄ and R₅ are hydrogen atoms, and in the formula (1), R₃ may be a benzyloxy group. In another embodiment, R₂ is a methoxy group, R₃ is a hydroxyl group, and R₁, R₄ and R₅ are a hydrogen atom, and in the formula (1), R₃ may be a benzyloxy group. In another embodiment, in formulas (1) and (2), R₂ is a hydroxyl group, R₃ is a methoxy group, and R₁, R₄ and R₅ are hydrogen atoms, and in formula (1), R₂ may be a benzyloxy group. In another embodiment, in formulas (1) and (2), R₁ is a hydroxyl group, and R₂, R₃, R₄ and R₅ are hydrogen atoms, and in formula (1), R₁ may be a benzyloxy group. In another embodiment, In another embodiment, in formula (1) and (2), R₁ is a hydroxyl group, R₃ is a methoxy group, and R₂, R₄ and R₅ are hydrogen atoms, and in formula (1), R₁ may be a benzyloxy group. In another embodiment, in formulas (1) and (2), R₁ is a hydroxyl group, R₃ is a methyl group, and R₂, R₄ and R₅ are hydrogen atoms, and in formula (1) R₁ may be a benzyloxy group. In another embodiment, in formulas (1) and (2) R₂ and R₃ are combined together to denote a methylene dioxy group, and R₁, R₄ and R₅ are hydrogen atoms. In another embodiment, in formulas (1) and (2) R₂ is a methyl group, R₃ is a methoxy group, and R₁, R₄ and R₅ are hydrogen atoms. In another embodiment, in formulas (1) and (2) R₂ is a methyl group, R₃ is a hydroxyl group, and R₁, R₄ and R₅ are hydrogen atoms, and in formula (1) R₃ may be a benzyloxy group. In another embodiment, in formulas (1) and (2) R₂ is a hydroxyl group, R₃ is a methyl group, and R₁, R₄ and R₅ are hydrogen atoms, and in formula (1) R₂ may be a benzyloxy group.

In the reductive alkylating reaction, one embodiment is to conduct the reaction in the presence of a catalyst, including hydrogenation catalysts, also including palladium carbon catalysts or platinum carbon catalysts. In one embodiment this reaction is conducted in a solvent, including those solvents such as alcohol or water-alcohol.

In another embodiment, in formulas (1) and (2) R₁, R₂, R₃, R₄ and R₅ are independently a hydrogen atom, a hydroxyl-group, a methoxy group, and a methyl group. In formula (1) one of R₁, R₂, R₃, R₄ and R₅ may independently a benzyloxy group. In one embodiment, R₁ and R₂; or R₂ and R₃ may form a methylene dioxy group.

In a process for producing 3-(3-hydroxy-4-methoxyphenyl)-3-methylbutylaldehyde where a carboxyl group in 3-(3-hydroxy-4-methoxyphenyl)-3-methylbutyric acid is converted to a formyl group, in one embodiment, the 3-(3-hydroxy-4-methoxyphenyl)-3-methylbutyric acid is obtained by converting a halogen atom in 3-(3-halogeno-4-methoxyphenyl)-3-methylbutyric acid to a hydroxyl group; or converting a halogen atom in 3-(3-halogeno-4-methoxyphenyl)-3-methylbutyric acid to a hydroxyl group, where in one embodiment 3-(3-halogeno-4-methoxyphenyl)-3-methylbutyric acid is obtained reacting 2-halogenoanisole with 3-methylcrotonic acid. In one embodiment the halogen atom in 3-(3-halogeno-4-methoxyphenyl)-3-methylbutyric acid is chlorine or bromine.

The reaction with 3-methylcrotonic acid is preferably conducted in the presence of an acid. To convert a carboxyl group into a formyl group can be accomplished by reducing a carboxylic acid to an aldehyde; or converting a carboxyl group into a hydroxymethyl group, and thereafter converting the hydroxymethyl into a formyl group.

The 3-(3-hydroxy-4-methoxyphenyl)-3-methylbutylaldehyde can be reductively alkylated with aspartame to produce N-[N-[3-(3-hydroxy-4-methoxyphenyl)-3-methylbutyl]-L-α-aspartyl]-L-phenylalanine 1-methyl ester.

The invention also provides a benzene derivative represented by formula (3):

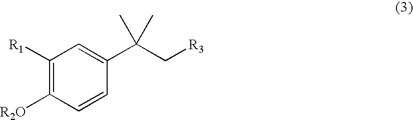

wherein in formula (3) R₁ may be a hydroxyl group, a halogen atom, or a lower alkyloxy group having 1 to 4 carbon atoms, R₂ may be a lower alkyl group having 1 to 4 carbon atoms, and R₃ may be a carboxyl group, a formyl group, or a hydroxymethyl group, provided that the compounds where R₁ is a chlorine atom or a bromine atom, and R₃ is a formyl group, are excluded. Examples of such benzene derivatives includes

3-(3-hydroxy-4-methoxyphenyl)-3-methylbutylaldehyde;

3-(3-chloro-4-methoxyphenyl)-3-methylbutyric acid;

3-(3-bromo-4-methoxyphenyl)-3-methylbutyric acid;

3-(3-hydroxy-4-methoxyphenyl)-3-methylbutyric acid; and

3-(3-hydroxy-4-methoxyphenyl)-3-methyl-1-butanol.

The invention also provides a process for producing N-[N-[3-(3-hydroxy-4-methoxyphenyl)-3-methylbutyl]-L-α-aspartyl]-L-phenylalanine 1-methyl ester by crystallizing N-[N-[3-(3-hydroxy-4-methoxyphenyl)-3-methylbutyl]-L-α-aspartyl]-L-phenylalanine 1-methyl ester containing impurity to a crystallization step to crystallize said compound.

In the process described above, where aspartame is dissolved in a solution, the aldehyde is added to the solution and dissolved, followed by the catalyst where the reaction is conducted under hydrogen gas, with stirring. After the reaction is complete, the catalyst is removed, for example, by filtration and the filtrate may be concentrated to obtain aspartyl dipeptide ester derivative, which can further be purified by common purification procedures, such as re-crystallization.

The following reaction process 3 can be used to produce the aldehyde represented by the general formula (1):

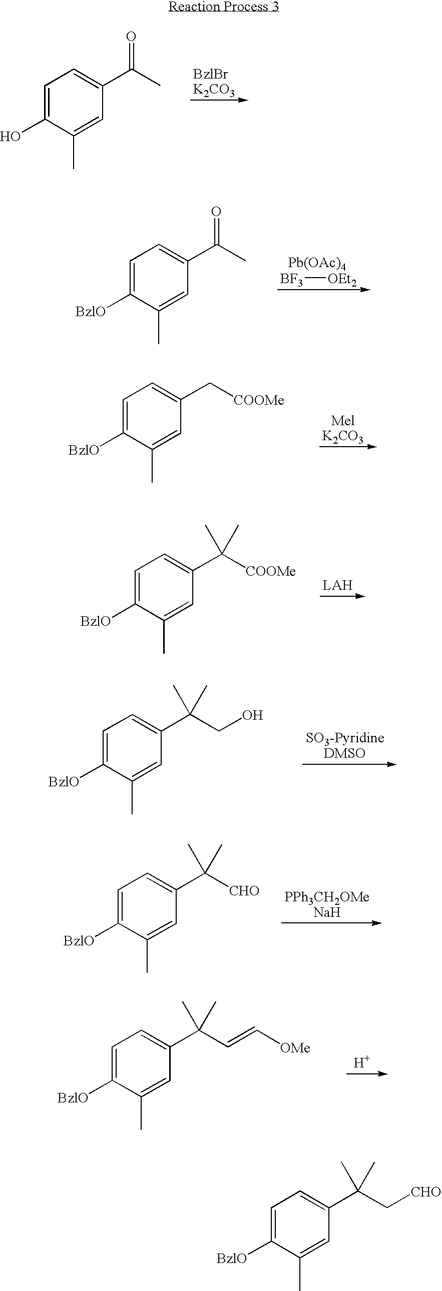

The solvent used in the reaction can be any solvent provided it does not react with the starting material, catalyst and the product. For example, an homogeneous organic solvent can be used to dissolve aspartame and the aldehyde, a mixture of solvents or a mixture of one ore more solvents with water can be used. For example, alcohols such as methanol and ethanol, tetrahydrofuran, acetonitrile, dimethylformamide and the like may be used. Preferably, an alcohol such as methanol, or water-containing alcohol such as water-containing methanol may be employed.

Suitable hydrogenation catalysts, if used, include those catalyst such as palladium, platinum, nickel and rhodium based catalysts. Preferably, palladium carbon, platinum carbon, rhodium carbon, Raney Nickel and the like are employed, more preferably palladium carbon and platinum carbon are used.

The reductive alkylation reaction can be conducted by hydrogenation, preferably under hydrogen pressure, for example, pressure of from about 0.1 to about 1.0 Mpa. The reaction temperature for the reductive alkylation reaction can vary, but to limit secondary reactions and to promote the desired reaction a temperature range of from about 15 to about 50° C. may be used. Preferably, the reaction is performed for about 2 to about 72 hours.

The molar ratio of aspartame to the aldehyde are preferably from about 0.5 to about 1.5 moles of aspartame per 1 mole of the aldehyde.

In the production of 3-(3-hydroxy-4-methoxyphenyl)-3-methylbutylaldehyde, the reaction of 2-halogenoanisole, such as 2-chloroanisole, 2-bromoanisole and the like, with 3-methylcrotonic acid, is preferably conducted without a solvent or in an organic solvent, preferably in the presence of an acid. In the event an organic solvent is employed, it should not react with the starting materials or products, such organic solvents include, for example, methylene chloride, chloroform, nitrobenzene and the like.

If an acid is employed, a proton acid (H⁺), such as sulfuric acid, para (p−)toluenesulfonic acid and hydrogen chloride, a Lewis acid (L.A.), such as aluminum chloride and titanium tetrachloride, and the like may be employed. Multiple acids can also be employed. Preferably, sulfuric acid, aluminum chloride and titanium tetrachloride are employed. In one embodiment, a proton acid may be combined with a Lewis acid, for example the combination of hydrogen chloride with aluminum chloride. The acid may be fixed onto the surface of the solid phase. Any amount of acid may be employed. An excess of acid to the 3-methylcrotonic acid may be employed to shorten the reaction time. From the economical point of view, preferably 5 molar equivalents or less, more preferably 3 molar equivalents or less, and further more preferably 0.1 to 3 molar equivalents, of the acid to the 3-methylcrotonic acid may be employed.

The amount of 2-halogenoanisole to 3-methylcrotonic acid, includes, but is not limited to, about 0.5 molar equivalents or more, more preferably 1 molar equivalent or more, and more preferably 1 to 10 molar equivalents, of 2-halogenoanisole to the 3-methylcrotonic acid.

The temperature for the reaction can be any temperature, however, the higher the reaction temperature, the more secondary reactions, and at a low temperature the reaction speed becomes too slow. Accordingly, a temperature range of about 20 to about 180° C. and more preferably about 30 to about 100° C. may be employed.

As described above, in case that 3-(3-hydroxy-4-methoxyphenyl)-3-methylbutyric acid is produced from the 3-(3-halogeno-4-methoxyphenyl)-3-methylbutyric acid obtained in the first stage, a reaction for converting the halogen atom substituted at the 3-position of the phenyl group into a hydroxyl group may be employed. For example, 3-(3-halogeno-4-methoxyphenyl)-3-methylbutyric acid can be heated in the presence of a copper catalyst in the alkaline aqueous solution, to convert the halogen atom into a hydroxyl group. The alkaline material employed may be a metal hydroxide, such as sodium hydroxide, potassium hydroxide and the like. The amount of the alkaline material used can be any amount, preferably from about 1 to about 10 moles to 1 mole of 3-(3-halogeno-4-methoxyphenyl)-3-methylbutyric acid.

The reaction temperature of converting to a hydroxyl group can be any temperature, but the higher the reaction temperature the more secondary reactions, and at a low temperature, the reaction speed becomes too slow. Accordingly, preferably the temperature is from about 100 to 250° C., and preferably from about 150 to about 200° C.

The copper catalyst includes those that may release univalent or divalent copper ion in the aqueous solution. For example, copper oxide(I), copper oxide(II), sulphate of copper(II), and the like Preferably, sulphate of copper(II) is employed.

In order to produce the aldehyde from 3-(3-hydroxy-4-methoxyphenyl)-3-methylbutyric acid the carboxylic acid is reduced into 3-(3-hydroxy-4-methoxyphenyl)-3-methylbutylaldehyde. The reduction can be effectuated as described in on the process described in Chemistry Letters, pp. 1143-1144 (1998). This process includes reducing 3-(3-hydroxy-4-methoxyphenyl)-3-methylbutyric acid with hydrogen in the organic solvent, with the addition of pivalic acid anhydride, palladium acetate and triphenylphosphine derivative. The organic solvent includes, but not limited to, acetone, tetrahydrofuran, toluene and the like. The amount employed of pivalic acid anhydride, equimolecular or more of the compound to 3-(3-hydroxy-4-methoxyphenyl)-3-methylbutyric acid, may be employed. For example, from about 1 to about 5 moles of the compound to 1 mole of 3-(3-hydroxy-4-methoxyphenyl)-3-methylbutyric acid may be employed. As triphenylphosphine derivative, triphenylphosphine and tritolylphosphine may be preferably employed. The palladium acetate and triphenylphosphine derivative can be used as a catalyst.

The amount of palladium acetate may be used in an amount of from about 0.1 to about 5 moles % and preferably from about 0.5 to about 3 mole % relative to the substrate. The triphenylphosphine derivative may be used in an amount of from about 5 times mole % or more and preferably from about 5 to 7 times mole % relative to the palladium acetate. The reaction temperature, includes but is not limited to from about 40 to about 100° C., and preferably from about 60 to about 80° C.

The carboxyl group can be completely reduced to a hydroxymethyl group, and thereafter oxidized partially to be able to produce the above aldehyde derivative. The reduction-partial oxidation reaction can be conducted as described, for example, in Journal of Organic Chemistry, vol. 48, No. 25, 5043-5048, 1983.

In the reduction from 3-(3-halogeno-4-methoxyphenyl)-3-methylbutyric acid, 3-(3-halogeno-4-methoxyphenyl)-3-methylbutylaldehyde, or 3-(3-halogeno-4-methoxyphenyl)-3-methyl-1-butanol can also be produced. In the production of N-[N-[3-(3-hydroxy-4-methoxyphenyl)-3-methylbutyl]-L-α-aspartyl]-L-phenylalanine 1-methyl ester from 3-(3-hydroxy-4-methoxyphenyl)-3-methylbutylaldehyde, a reductive alkylation of the aldehyde with α-L-aspartyl-L-phenylalanine methyl ester (aspartame) under hydrogenation condition. Specifically, in the solvent which can dissolve the starting materials, for example, alcohol, water-containing alcohol, or the like, in the presence of the catalyst for a reductive alkylation, for example, a catalyst such as palladium based catalyst, a reductive alkylation reaction is conducted with hydrogen, more preferably, under suitable or effective reaction temperature and pressure to be able to produce the above object compound. There are no particular limitations on the reaction solvent, as long as it is inactive to the substrate, a catalyst and the product. A homogeneous organic solvent which can dissolve aspartame and the aldehyde, a mixture of solvents, or a mixture of solvent(s) and water may be used. Suitable organic solvents include, but are not limited to, alcohols such as methanol and ethanol, tetrahydrofuran, acetonitrile, dimethylformamide and the like. Preferably, an alcohol such as methanol, or water-containing alcohol such as water-containing methanol is used.

The N-[N-[3-(3-hydroxy-4-methoxyphenyl)-3-methylbutyl]-L-α-aspartyl]-L-phenylalanine 1-methyl ester can be purified by crystallization. The N-[N-[3-(3-hydroxy-4-methoxyphenyl)-3-methylbutyl]-L-α-aspartyl]-L-phenylalanine 1-methyl ester can be in a solution form, a solid form, or a form of an intermediate stage therebetween.

In order to obtain the object compound of N-[N-[3-(3-hydroxy-4-methoxyphenyl)-3-methylbutyl]-L-α-aspartyl]-L-phenylalanine 1-methyl ester as the solid form, a reaction solution may be purified with a silica gel column chromatography and the obtained fractions containing the N-[N-[3-(3-hydroxy-4-methoxyphenyl)-3-methylbutyl]-L-α-aspartyl]-L-phenylalanine 1-methyl ester is concentrated to solidification. However, this process is high in cost, and problematic in treatment of the used silica gel, and therefore is undesirable for industrial operations. Further, in the powder X-ray analysis obtained by the present inventors, the solid obtained in this process is in the amorphous state, and is also undesirable in stability.

The object compound described above is useful as a sweetener used for a food, a pharmaceutical product and the like, and therefore the compound in its final form should be at a high purity and excellent in stability.

When the N-[N-[3-(3-hydroxy-4-methoxyphenyl)-3-methylbutyl]-L-α-aspartyl]-L-phenylalanine 1-methyl ester to be purified is in the liquid state, the insoluble material undesirable for the crystallization step, for example, the used catalyst or the like, is in advance by separation with filtration. The thus obtained solution is subjected to the crystallization under a condition suitable for the crystallization of the N-[N-[3-(3-hydroxy-4-methoxyphenyl)-3-methylbutyl]-L-α-aspartyl]-L-phenylalanine 1-methyl ester. For example, in case that the solution can be concentrated under reduced pressure or after such concentration, the crystallization solvent can be added to concentrated solution followed by cooling or concentrating to crystallize the object compound.

When the object compound is in a solvent undesirable for the crystallization, the solvent can be removed by distillation and thereafter, a suitable crystallization to dissolve the object compound is employed. The crystallization process can also be performed on the material not containing a solvent. When the solvent is a nonpolar solvent, such as aldehyde and its derivativean additional step of extraction is preferably included. The extraction can be performed before the crystallization step, during the crystallization step or after the crystallization step. The extraction is preferably conducted where the crystals are dissolved therein. However, it may be conducted in the state where the crystals are not dissolved therein completely, that is in the slurry state.

The extraction may be performed by adding directly to the reaction solution or to the solution which has been concentrated partially, a suitable extraction solvent and water, if necessary, are added to dissolve the impurity in the solvent; and then to remove it from the aqueous layer. An aqueous layer at the time of such extraction with water, may contain, for example, at least one solvent selected from the group consisting of methanol, ethanol, isopropyl alcohol, tetrahydrofuran, acetonitrile, acetic acid and ethyl acetate, which is a solvent usable for the reductive alkylation reaction, and which does not prevent separation at the extraction step.

The solvent used for the extraction with solvent in the present invention is a solvent which can not be mixed with water homogeneously. For example, at least one solvent selected from toluene, diethyl ether, chloroform, dichloromethane, hexane, ethyl acetate, propyl acetate, isopropyl acetate and butyl acetate, can be used. More preferably, toluene may be employed.

For the solvent used for the crystallization step in the present invention, at least one solvent selected from a lower alcohol such as methanol, ethanol, isopropyl alcohol and the like, ketones such as acetone, methyl ethyl ketone, methyl isobutyl ketone and the like, esters such as methyl acetate, ethyl acetate, propyl acetate, isopropyl acetate, butyl acetate and the like, tetrahydrofuran, acetonitrile and toluene, or a mixed solvent of at least one solvent of these organic solvents with water, are preferably employed. More preferably, methanol, ethanol, acetone and water can be employed. Further, preferably, methanol, ethanol and/or acetone which are good solvents can be employed properly in combination with water which is a poor solvent.

The process can be performed, for example, by combining partially or completely removing the solvent by concentration, addition of the crystallization solvent and crystallization under cooling. To crystallize N-[N-[3-(3-hydroxy-4-methoxyphenyl)-3-methylbutyl]-L-α-aspartyl]-L-phenylalanine 1-methyl ester, which obtained in the process for reductively alkylating aspartame and 3-(3-hydroxy-4-methoxyphenyl)-3-methylbutylaldehyde in a mixed solvent of methanol and water (mixing ratio of 3:2 v/v), the catalyst is separated by filtration, toluene is added for extraction, and the impurity is removed. The aqueous layer obtained is concentrated under reduced pressure to partially remove methanol, and cooled to crystallize the object compound for separation of the crystals.

The crystals can be separated by filtration, centrifugal separation or other methods of separating commonly employed in the art. To separate the crystals, the crystals can be dried where necessary. For example, a usual method such as drying under reduced pressure, through-flow drying, and the like can be employed.

The crystallization solvent, composition of solvent, quantity employed of solvent, method of crystallization will vary and can be performed according to procedures known in the art. For example, when crystallizing by cooling the object compound in the solution is in a concentration of from about 0.5 to about 20 g/dl, preferably from about 1 to about 15 g/dl, and more preferably about 2 to about 10 g/dl. When the concentration is too low the yield is lowered, and when the concentration is too high the purity is lowered. The temperature for crystallization is, for example, from about 40 to about 80° C., and preferably-from about 50 to about 70° C. When the temperature is too high decomposition of the object compound, distillation (vaporization) of the crystallization solvent and other negative effects may be observed. To finish crystallization, the temperature may be, for example, at a temperature where the solution is not solidified; preferably from about 20 to about 5° C., and more preferably from about 15 to about 5° C., where the cooling time can be conducted at any suitable time.

Crystals of N-[N-[3-(3-hydroxy-4-methoxyphenyl)-3-methylbutyl]-L-α-aspartyl]-L-phenylalanine 1-methyl ester exhibit the following physical properties: peaks of diffractive X-ray in at least diffraction angles of 8.3°, 19.5° and 21.2° (2θ, CuK αray) when determined in the powder X-ray diffractometry.

The present invention also provides food, drinks or other edible compounds, to which are desired to be sweetened have crystals of N-[N-[3-(3-hydroxy-4-methoxyphenyl)-3-methylbutyl]-L-α-aspartyl]-L-phenylalanine 1-methyl ester, which may further have in order to give stability a carrier, a thickening agent (viscosity improver), a bulking agent, and/or an excipient (diluting agent) for sweeteners, where necessary, can be employed.

The present invention also provides a process for imparting a sweet taste by adding the crystals as described above, or including said crystals in a product such as a food and drink in need of a sweet taste; or an intermediate product.

Examples of such food and drinks in need of a sweetener include, but are not limited to, confectionary (frozen dessert, jelly, cake, candy (sweet or the like), bread, chewing gums, hygiene product, cosmetics (including an oral composition such as dentifrice and others), a pharmaceutical product, and various veterinary product for animal other than for humans or the like.

### EXAMPLES

The present invention will be explained further in detail with reference to the following Examples.

### Example 1

Synthesis of 3-(3-chloro-4-methoxyphenyl)-3-methylbutyric acid

To 2-chloroanisole (100.0 g), 95% sulfuric acid (72.4 g) and 3-methylcrotonic acid (35.1 g) were added and the mixture was stirred under heating at 70° C. for 67 hours, and thereafter the reaction was stopped by addition of water (200 ml) thereto. Thus obtained mixture was extracted with methylene chloride (400 ml), and to the separated organic layer 1 normal (1N) caustic soda aqueous solution (200 ml) was added for further extraction. To the separated aqueous layer hydrochloric acid (HCl) was added to make it acidification, and the mixture was extracted with methylene chloride and the solvent therein was distilled off. Thus obtained residue was re-crystallized with ethyl acetate and hexane to obtain 3-(3-chloro-4-methoxyphenyl)-3-methylbutyric acid (10.9 g, yield of 12.7% to 3-methylcrotonic acid).

¹H NMR (CDCl₃) δ: 1.43 (s, 6H), 2.61 (s, 2H), 3.87 (s, 3H), 6.86 (d, J=8.6 Hz, 1H), 7.10-7.23 (m, 1H), 7.35 (d, J=2.4 Hz, 1H).

ESI-MS;

Calculation: C₁₂H₁₅³⁵ClO₃=242.3, Analysis: 241.3(MH^{-).}

### Example 2

Synthesis of 3-(3-bromo-4-methoxyphenyl)-3-methylbutyric acid

To 2-bromoanisole (50 g), 95% sulfuric acid (27.6 g) and 3-methylcrotonic acid (5.35 g) were added and the mixture was stirred under heating at 70° C. for 27 hours, and thereafter the reaction was stopped by addition of water (100 ml) thereto. Thus obtained mixture was extracted with methylene chloride (100 ml), and to the separated organic layer 1 normal (1N) caustic soda aqueous solution (100 ml) was added for further extraction. To the separated aqueous layer hydrochloric acid (HCl) was added to make it acidification, and the mixture was extracted with methylene chloride and the solvent therein was distilled off. Thus obtained residue was re-crystallized with ethyl acetate and hexane to obtain 3-(3-bromo-4-methoxyphenyl)-3-methylbutyric acid (3.1 g, yield of 20.3% to 3-methylcrotonic acid).

### Example 3

Synthesis of 3-(3-bromo-4-methoxyphenyl)-3-methylbutyric acid

A mixture of 2-bromoanisole (102.4 g) and 3-methylcrotonic acid (16.1 g) was stirred for mixing, and aluminum chloride (23.5 g) was added thereto. The mixture was stirred under heating at 70° C. for 5 hours, and thereafter the reaction solution was cooled to a room temperature. After that, the reaction was stopped by addition of 6 normal (6N) hydrochloric acid (300 ml) thereto. Thus obtained mixture was extracted with toluene (300 ml), and the separated organic layer was further extracted with 1 normal (1N) caustic soda aqueous solution (500 ml). Subsequently, to the separated aqueous layer 6N-hydrochloric acid (HCl) was added to make it acidification, and the mixture was extracted with toluene (600 ml) and the organic layer was concentrated under reduced pressure to obtain crude crystals thereof. Thus obtained crude crystals were re-crystallized with ethyl acetate and hexane to obtain 3-(3-bromo-4-methoxyphenyl)-3-methylbutyric acid (20.7 g, yield of 45% to 3-methylcrotonic acid).

¹HNMR (CDCl₃) δ: 1.43 (s, 6H), 2.61 (s, 2H), 3.87 (s, 3H), 6.84 (d, J=8.7 Hz, 1H), 7.20-7.29 (m, 1H), 7.52 (d, J=2.4 Hz, 1H).

ESI-MS;

Calculation: C₁₂H₁₅⁷⁹BrO₃=286.2, Analysis: 285.2(MH^{-).}

### Example 4

Synthesis of 3-(3-hydroxy-4-methoxyphenyl)-3-methylbutyric acid

Into a stainless steel reaction vessel withstand pressure, 3-(3-bromo-4-methoxyphenyl)-3-methylbutyric acid (18.8 g), cupric sulfate 5 hydrate (8.2 g), sodium hydroxide (48.16 g) and distilled water (159 ml) in these order were filled, and the mixture was stirred for 1 hour at room temperature and then for 10 hours under heating at 160° C., and then cooled to a room temperature.

To thus obtained reaction solution, hydrochloric acid (HCl) was added to make it acidification, and the mixture was extracted with ethyl acetate and the organic layer was washed with sodium chloride saturated aqueous solution. The solvent therein was distilled off under reduced pressure to obtain crude crystals. Thus obtained crude crystals were re-crystallized with ethyl acetate and hexane to obtain 3-(3-hydroxy-4-methoxyphenyl)-3-methylbutyric acid (10.5 g, yield of 72%).

¹HNMR (CDCl₃) δ: 1.42 (s, 6H), 2.60 (s, 2H), 3.86 (s, 3H), 6.78 (d, J=8.5 Hz, 1H), 6.84 (dd, J=2.2, 8.5 Hz, 1H), 6.95 (d, J=2.2 Hz, 1H).

ESI-MS;

Calculation: C₁₂H₁₆O₄=224.3, Analysis: 223.2(MH⁻).

### Example 5

Synthesis of 3-(3-hydroxy-4-methoxyphenyl)-3-methylbutyl aldehyde

Into a chemical reactor for hydrogen addition (hydrogenation) under elevated pressure, 3-(3-hydroxy-4-methoxyphenyl)-3-methylbutyric acid (13.6 g), pivalic acid anhydride (22.8 g) and acetone (100 ml) were filled, and thereafter the mixture was bubbled with nitrogen gas for 30 minutes to substitute nitrogen gas completely for the gas in the system of reaction, whereby the system was filled with nitrogen gas. Subsequently, palladium acetate (137 mg) produced previously and tri-p-tolylphosphine (930 mg) in tetrahydrofuran solution (50 ml) were added thereto, and the mixture was stirred under hydrogen pressure of 5 MPa at 80° C. for 24 hours for reaction.

From the reaction solution, acetone was distilled off, and thus obtained residue was purified-by the use of column chromatography to obtain 3-(3-hydroxy-4-methoxyphenyl)-3-methylbutylaldehyde (10.2 g, yield: 80%).

¹HNMR (CDCl₃) δ: 1.41 (s, 6H), 2.61 (d, J=3.0 Hz, 2H), 3.87 (s, 3H), 6.72-6.84 (m, 2H), 6.98 (d, J=1.9 Hz, 1H), 9.49 (t, J=3.0 Hz, 1H).

ESI-MS;

Calculation: C₁₂H₁O₃=208.3, Analysis: 207.2(MH⁻).

### Example 6

Synthesis of N-[N-[3-(3-hydroxy-4-methoxyphenyl)-3-methylbutyl]-L-α-aspartyl]-L-phenylalanine 1-methyl ester

Aspartame (5.89 g, 20.0 mmol) and 3-(3-hydroxy-4-methoxyphenyl)-3-methylbutylaldehyde (3.96 g, 19.0 mmol) were added to 80% methanol aqueous solution (200 ml), and the mixture was stirred at 40° C. in a short time. To this solution, 10% palladium carbon in the water content of 50% (1.78 g) was added, and the mixture was stirred under hydrogen atmosphere of normal pressure (0.1 MPa) at 40° C. for 40 hours. Thus obtained reaction solution was filtrated to remove the catalyst, and the filtrate was subjected to the high performance liquid chromatography (HPLC) for determination and thereby the title compound (5.38 g, 10.7 mmol, 56.6%) was produced.

### Example 7

Production of N-[N-[3-(4-methoxyphenyl)-3-methylbutyl]-L-α-aspartyl]-L-phenylalanine 1-methyl ester

Aspartame (5.89 g, 20.0 mmol) and 3-(4-methoxyphenyl)-3-methylbutylaldehyde (3.65 g, 19.0 mmol) were added to 80% methanol aqueous solution (200 ml), and the mixture was stirred at 40° C. in a short time. To this solution, 10% palladium carbon in the water content of 50% (1.78 g) was added, and the mixture was stirred under hydrogen atmosphere of normal pressure (0.1 MPa) at 40° C. for 40 hours for reaction. Thus obtained reaction solution was filtrated to remove the catalyst, and the filtrate was subjected to the high performance liquid chromatography (HPLC) for determination and thereby the title compound (6.45 g, 13.7 mmol, 72.2%) was produced.

### Example 8

Production of N-[N-[3-(4-hydroxyphenyl)-3-methylbutyl]-L-α-aspartyl]-L-phenylalanine 1-methyl ester

Aspartame (5.89 g, 20.0 mmol) and 3-(4-hydroxyphenyl)-3-methylbutylaldehyde (3.38 g, 19.0 mmol) were added to 80% methanol aqueous solution (200 ml), and the mixture was stirred at 40° C. in a short time. To this solution, 10% palladium carbon in the water content of 50% (1.78 g) was added, and the mixture was stirred under hydrogen atmosphere of normal pressure (0.1 MPa) at 40° C. for 40 hours for reaction. Thus obtained reaction solution was filtrated to remove the catalyst, and the filtrate was subjected to the HPLC for determination and thereby the title compound (5.59 g, 12.3 mmol, 64.5%) was produced.

### Example 9

Production of N-[N-[3-(3-methoxy-4-hydroxyphenyl)-3-methylbutyl]-L-α-aspartyl]-L-phenylalanine 1-methyl ester

Aspartame (5.89 g, 20.0 mmol) and 3-(3-methoxy-4-hydroxyphenyl)-3-methylbutylaldehyde (3.96 g, 19.0 mmol) were added to 80% methanol aqueous solution (200 ml), and the mixture was stirred at 40° C. in a short time. To this solution, 10% palladium carbon in the water content of 50% (1.78 g) was added, and the mixture was stirred under hydrogen atmosphere of normal pressure (0.1 MPa) at 40° C. for 40 hours for reaction. Thus obtained-reaction solution was filtrated to remove the catalyst, and the filtrate was subjected to the HPLC for determination and thereby the title compound (5.74 g, 11.8 mmol, 62.2%) was produced.

### Example 10

Production of N-[N-[3-(2-hydroxyphenyl)-3-methylbutyl]-L-α-aspartyl]-L-phenylalanine 1-methyl ester

Aspartame (5.89 g, 20.0 mmol) and 3-(2-hydroxyphenyl)-3-methylbutylaldehyde (3.39 g, 19.0 mmol) were added to 80% methanol aqueous solution (200 ml), and the mixture was stirred at 40° C. in a short time. To this solution, 10% palladium carbon in the water content of 50% (1.78 g) was added, and the mixture was stirred under hydrogen atmosphere of normal pressure (0.1 MPa) at 40° C. for 40 hours for reaction. Thus obtained reaction solution was filtrated to remove the catalyst, and the filtrate was subjected to the HPLC for determination and thereby the title compound (5.81 g, 12.3 mmol, 64.5%) was produced.

### Example 11

Production of N-[N-[3-(2-hydroxy-4-methoxyphenyl)-3-methylbutyl]-L-α-aspartyl]-L-phenylalanine 1-methyl ester

Aspartame (5.89 g, 20.0 mmol) and 3-(2-hydroxy-4-methoxyphenyl)-3-methylbutylaldehyde (3.96 g, 19.0 mmol) were added to 80% methanol aqueous solution (200 ml), and the mixture was stirred at 40° C. in a short time. To this solution, 10% palladium carbon in the water content of 50% (1.78 g) was added, and the mixture was stirred under hydrogen atmosphere of normal pressure (0.1 MPa) at 40° C. for 40 hours for reaction. Thus obtained reaction solution was filtrated to remove the catalyst, and the filtrate was subjected to the HPLC for determination and thereby the title compound (4.21 g, 8.38 mmol, 44.1%) was produced.

### Example 12

Production of N-[N-[3-(2-hydroxy-4-methylphenyl)-3-methylbutyl]-L-α-aspartyl]-L-phenylalanine 1-methyl ester

Aspartame (5.89 g, 20.0 mmol) and 3-(2-hydroxy-4-methylphenyl)-3-methylbutylaldehyde (3.65 g, 19.0 mmol) were added to 80% methanol aqueous solution (200 ml), and the mixture was stirred at 40° C. in a short time. To this solution, 10% palladium carbon in the water content of 50% (1.78 g) was added, and the mixture was stirred under hydrogen atmosphere of normal pressure (0.1 MPa) at 40° C. for 40 hours for reaction. Thus obtained reaction solution was filtrated to remove the catalyst, and the filtrate was subjected to the HPLC for determination and thereby the title compound (4.17 g, 8.57 mmol, 45.1%) was produced.

### Example 13

Production of N-[N-[3-(3,4-methylenedioxyphenyl)-3-methylbutyl]-L-α-aspartyl]-L-phenylalanine 1-methyl ester

Aspartame (5.89 g, 20.0 mmol) and 3-(3,4-methylenedioxyphenyl)-3-methylbutylaldehyde (3.92 g, 19.0 mmol) were added to 80% methanol aqueous solution (200 ml), and the mixture was stirred at 40° C. in a short time. To this solution,10% palladium carbon in the water content of 50% (1.78.g) was added, and the mixture was stirred under hydrogen atmosphere of normal pressure (0.1 MPa) at 40° C. for 40 hours for reaction. Thus obtained reaction solution was filtrated to remove the catalyst, and the filtrate was subjected to the HPLC for determination and thereby the title compound (6.4 g, 13.2 mmol, 69.7%) was produced.

### Example 14

Production of N-[N-[3-(3-methyl-4-methoxyphenyl)-3-methylbutyl]-L-α-aspartyl]-L-phenylalanine 1-methyl ester

Aspartame (5.89 g, 20.0 mmol) and 3-(3-methyl-4-methoxyphenyl)-3-methylbutylaldehyde (3.92 g, 19.0 mmol) were added to 80% methanol aqueous solution (200 ml), and the mixture was stirred at 40° C. in a short time. To this solution, 10% palladium carbon in the water content of 50% (1.78 g) was added, and the mixture was stirred under hydrogen atmosphere of normal pressure (0.1 MPa) at 40° C. for 40 hours for reaction. Thus obtained reaction solution was filtrated to remove the catalyst, and the filtrate was subjected to the HPLC for determination and thereby the title compound (6.06 g, 12.5 mmol, 66.0%) was produced.

### Example 15

Production of N-[N-[3-(3-methyl-4-hydroxyphenyl)-3-methylbutyl]-L-α-aspartyl]-L-phenylalanine 1-methyl ester

Aspartame (5.89 g, 20.0 mmol) and 3-(3-methyl-4-hydroxyphenyl)-3-methylbutylaldehyde (3.65 g, 19.0 mmol) were added to 80% methanol aqueous solution (200 ml), and the mixture was stirred at 40° C. in a short time. To this solution, 10% palladium carbon in the water content of 50% (1.78 g) was added, and the mixture was stirred under hydrogen atmosphere of normal pressure (0.1 MPa) at 40° C. for 40 hours for reaction. Thus obtained-reaction solution was filtrated to remove the catalyst, and the filtrate was subjected to the HPLC for determination and thereby the title compound (5.65 g, 12.0 mmol, 63.2%) was produced.

### Example 16

Production of N-[N-[3-(3-hydroxy-4-methylphenyl)-3-methylbutyl]-L-α-aspartyl]-L-phenylalanine 1-methyl ester

Aspartame (5.89 g, 20.0 mmol) and 3-(3-hydroxy-4-methylphenyl)-3-methylbutylaldehyde (3.65 g, 19.0 mmol) were added to 80% methanol aqueous solution (200 ml), and the mixture was stirred at 40° C. in a short time. To this solution, 10% palladium carbon in the water content of 50% (1.78 g) was added, and the mixture was stirred under hydrogen atmosphere of normal pressure (0.1 MPa) at 40° C. for 40 hours for reaction. Thus obtained reaction solution was filtrated to remove the catalyst, and the filtrate was subjected to the HPLC for determination and thereby the title compound (5.84 g, 12.4 mmol, 65.5%) was produced.

### Example 17

Synthesis of N-[N-[3-(3-hydroxy-4-methoxyphenyl)-3-methylbutyl]-L-α-aspartyl]-L-phenylalanine 1-methyl ester, Crystallization thereof, and Separation of the crystals thereof

3-(3-Hydroxy-4-methoxyphenyl)-3-methylbutylaldehyde (6.677 g, 25.2 mmol) was dissolved in 80% methanol aqueous solution (272 ml), and aspartame (8.446 g, 27.8 mmol) was added thereto to prepare a slurry solution. 10% Palladium carbon in the water content of 50% (2.86 g) was added thereto under nitrogen stream, and thereafter substitution of hydrogen for the reaction system was performed and the mixture as it is was stirred at 25° C. for 24 hours. After substitution of nitrogen therefor, the catalyst was separated by filtration, and further washed with methanol (30 ml). After that, to the filtrate water (193 ml) was added, and thus obtained mixture was extracted with toluene (247.6 ml) twice. The separated methanol/water layer was concentrated under reduced pressure to approximately one second (1/2) of quantity thereof by weight. After that, the concentrated solution was cooled sequentially from 75° C. to 5° C. to precipitate crystals. Thus separated crystals were dissolved in 50% methanol aqueous solution (260 ml) at 75° C., and thus obtained solution was cooled to 5° C. to precipitate crystals. The crystals were dried under reduced pressure to obtain N-[N-[3-(3-hydroxy-4-methoxyphenyl)-3-methylbutyl]-L-α-aspartyl]-L-phenylalanine 1-methyl ester (8.46 g, 17.1 mmol, yield of 67.6% based on the aldehyde) in the white crystalline form.

(The purity in the HPLC analysis was not less than 98%.)

¹H NMR (DMSO-d₆) δ: 1.14 (brs, 6H), 1.54-1.68 (m, 2H), 2.04-2.22 (m, 3H), 2.24-2.34 (dd, 1H), 2.84-2.94 (dd, 1H), 3.00-3.08 (dd, 1H), 3.31-3.36 (m, 1H), 3.59 (s, 3H), 3.71 (s, 3H), 4.46-4.55 (m, 1H), 6.60-6.65 (dd, 1H), 6.73 (s, 1H), 6.80 (d, 1H), 7.10-7.28 (m, 5H), 8.45 (d, 1H), 8.75 (brs, 1H).

ESI-MS; 487.3 (MH⁺).

### Example 18

Crystallization of N-[N-[3-(3-hydroxy-4-methoxyphenyl)-3-methylbutyl]-L-α-aspartyl]-L-phenylalanine 1-methyl ester, and Separation of the crystals thereof

3-(3-Hydroxy-4-methoxyphenyl)-3-methylbutylaldehyde (0.460 g, 2.21 mmol) and aspartame (0.683 g, 2.32 mmol) were added to a mixed solvent (26 ml) of methanol and water (Mixing ratio of 3:2 v/v), and then the mixture was stirred at room temperature in a short time. 10% Palladium carbon in the water content of 50% (0.233 g) was added thereto, and the mixture was stirred under hydrogen atmosphere of normal pressure (0.1 MPa) at room temperature for 48 hours for reaction. Thus obtained reaction solution was heated up to 45° C. and stirred for 30 minutes. After that, thus obtained reaction solution was filtrated to remove the catalyst, and further, the catalyst was washed with methanol (10 ml). The filtrate and the wash solution were combined together to obtain a reaction solution (38 ml) containing N-[N-[3-(3-hydroxy-4-methoxyphenyl)-3-methylbutyl]-L-α-aspartyl]-L-phenylalanine 1-methyl ester (0.795 g, 1.63 mmol). This reaction solution was concentrated under reduced pressure at 40° C. up to approximately one fourth (1/4) of the volume thereof, and then to the concentrated solution while stirring at 50° C., methanol (1 ml) was added to precipitate crystals. This crystallization solution was cooled to 5° C. and allowed to stand overnight at the same temperature. The crystals were separated by filtration, and further washed with water (20 ml), and dried overnight under reduced pressure at room temperature to obtain crude crystals thereof (0.841 g, Content of the object compound: 0.513 g, Recovery rate: 65%). Thus obtained crude crystals were added to a mixed solvent (26 ml) of methanol and water (Mixing ratio of 3:2 v/v) and dissolved therein at 60° C. After that, the solution was cooled to 5° C. to precipitate crystals. The mixture was allowed to stand overnight at the same temperature, and then the crystals were separated by filtration, and washed with water in small quantities, and dried under reduced pressure at 40° C. for 4 hours to obtain N-[N-[3-(3-hydroxy-4-methoxyphenyl)-3-methylbutyl]-L-α-aspartyl]-L-phenylalanine 1-methyl ester (0.402 g, 0.826 mmol, Crystallization yield: 78%).

The purity in the high performance liquid chromatography (HPLC) analysis was not less than 97%.

### Example 19

Crystallization of N-[N-[3-(3-hydroxy-4-methoxyphenyl)-3-methylbutyl]-L-α-aspartyl]-L-phenylalanine 1-methyl ester, and Separation of the crystals thereof

3-(3-Hydroxy-4-methoxyphenyl)-3-methylbutylaldehyde (6.01 g, 28.8 mmol) and aspartame (9.89 g, 33.6 mmol) were added to a mixed solvent (330 ml) of methanol and water (Mixing ratio of 3:2 v/v), and then the mixture was stirred at 25° C. in a short time. 10% Palladium carbon in the water content of 50% (3.46 g) was added thereto, and the mixture was stirred under hydrogen atmosphere of normal pressure (0.1 MPa) at 25° C. for 47 hours for reaction. Thus obtained reaction solution was heated up to 45° C. and stirred for 30 minutes. After that, thus obtained reaction solution was filtrated to remove the catalyst, and further, the catalyst was washed with a mixed solvent (150 ml) of methanol and water (Mixing ratio of 3:2 v/v). The filtrate and the wash solution were combined together to obtain a reaction solution (439 g) containing N-[N-[3-(3-hydroxy-4-methoxyphenyl)-3-methylbutyl]-L-α-aspartyl]-L-phenylalanine 1-methyl ester (9.32 g, 19.2 mmol). To this reaction solution, toluene (288 ml) was added, and thus obtained solution was stirred at room temperature for 30 minutes to make layers separated therein. The toluene layer was removed therefrom, and thereby the aqueous layer (458 g) was obtained.

This aqueous layer was concentrated under reduced pressure at 40° C. up to an amount of residual solution of 224 g, and heated up to 75° C., and then cooled to 5° C. while stirring (Cooling speed: 10° C./hour) to precipitate crystals. Thus obtained mixture was stirred at the same temperature for 16 hours. After that, the crystals were separated by filtration, and further washed with water (20 ml) to obtain the wet crude crystals (14.0 g, Content of the object compound: 8.61 g, Recovery rate: 92%). To thus obtained wet crude crystals, a mixed solvent (900 ml) of methanol and water (Mixing ratio of 2:3 v/v) was added, and the crystals were dissolved therein at 75° C. The solution was cooled while stirring to 5° C. (Cooling speed: 10° C./hour) to precipitate crystals. The mixture was stirred at the same temperature for 65 hours, and thereafter the crystals were separated by filtration, and further washed with a mixed solvent (20 ml) of methanol and water (Mixing ratio: 2:3 v/v), and dried under reduced pressure at room temperature for 5 hours to obtain N-[N-[3-(3-hydroxy-4-methoxyphenyl)-3-methylbutyl]-L-α-aspartyl]-L-phenylalanine 1-methyl ester (6.15 g, 12.6 mmol, Crystallization yield: 71%). (The purity in the HPLC analysis was not less than 98%.)

### Example 20

Crystallization of N-[N-[3-(3-hydroxy-4-methoxyphenyl)-3-methylbutyl]-L-α-aspartyl]-L-phenylalanine 1-methyl ester and Separation of the crystals thereof

3-(3-Hydroxy-4-methoxyphenyl)-3-methylbutylaldehyde (0.423 g, 2.03 mmol) and aspartame (0.683 g, 2.32 mmol) were added to a mixed solvent (26 ml) of methanol and water (Mixing ratio of 3:2 v/v), and then the mixture was stirred at room temperature in a short time. 10% Palladium carbon in the water content of 50% (0.233 g) was added thereto, and the mixture was stirred under hydrogen atmosphere of normal pressure (0.1 MPa) at room temperature for 48 hours for reaction. Thus obtained reaction solution was heated up to 45° C. and stirred for 30 minutes. After that, thus obtained reaction solution was filtrated to remove the catalyst, and further, the catalyst was washed with methanol (10 ml). The filtrate and the wash solution were combined together to obtain a reaction solution (32.6 g)-containing N-[N-[3-(3-hydroxy-4-methoxyphenyl)-3-methylbutyl]-L-α-aspartyl]-L-phenylalanine 1-methyl ester (0.988 g, 2.03 mmol).

This reaction solution was concentrated under reduced pressure at 40° C. up to an amount of residual solution of 10.1 g, and heated up to 75° C., and then cooled to 5 ?C while stirring (Cooling speed: 10° C./hour) to precipitate crystals. Thus obtained mixture was stirred overnight at the same temperature. After that, the crystals were separated by filtration, and further washed with water (2.6 ml) to obtain the wet crude crystals (1.31 g, Content of the object compound: 0.909 g, Recovery rate: 92%). To thus obtained wet crude crystals, a mixed solvent (52 ml) of methanol and water (Mixing ratio of 1:1 v/v), and toluene (26 ml) were added. Thus obtained mixture was stirred at room temperature for 10 minutes to make layers separated therein. The toluene layer was removed, and thereby the aqueous layer was obtained, and then was concentrated under reduced pressure at 40° C. to obtain a concentrated solution (24.1 g). To this concentrated solution, methanol (4 ml) was added, and the insoluble material was dissolved therein at 75° C. Thus obtained solution was cooled to 5° C. while stirring (Cooling speed: 10° C./hour) to precipitate crystals. After the mixture was stirred for 40 hours at the same temperature, the crystals were separated by filtration, and further washed with a mixed solvent (3 ml) of methanol and water (Mixing ratio: 1:4 v/v), and dried under reduced pressure at room temperature for 5 hours to obtain N-[N-[3-(3-hydroxy-4-methoxyphenyl)-3-methylbutyl]-L-α-aspartyl]-L-phenylalanine 1-methyl ester (0.617 g, 1.268 mmol, Crystallization yield: 68%). (The purity in the HPLC analysis was not less than 98%.)

### Example 21

Physical properties on N-[N-[3-(3-hydroxy-4-methoxyphenyl)-3-methylbutyl]-L-α-aspartyl]-L-phenylalanine 1-methyl ester in the crystalline form

The physical properties on the crystals of the title compound obtained in the present invention were in the followings.

Differential thermal analysis:

Temperature range for the determination: 50-300° C.; Heating-up speed: 10° C./minute;

Melting point: 189° C.

Powder X-ray diffraction:

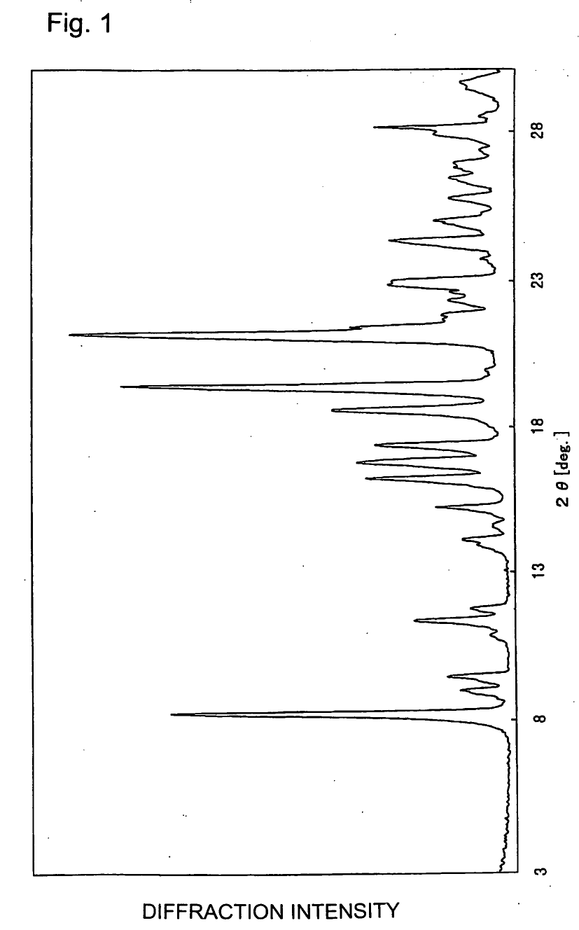

As shown in the FIG. 1, the characteristic peaks were observed (exhibited) in the diffraction-angles of 8.3°, 19.5° and 21.2° (2θ, CuK αray).

### Effect of Invention

According to the present invention, aspartame is alkylated reductively with 3-(phenyl with substituent group(s))-3-methylbutylaldehyde to be able to produce N-[N-[3-(phenyl with substituent group(s))-3-methylbutyl]-L-α-aspartyl]-L-phenylalanine 1-methyl ester derivative efficiently on an industrial scale, which is excellent as a sweetener having a high degree of sweetness and therefore is an objective compound described above.

Further, by using a novel 3-(3-hydroxy-4-methoxyphenyl)-3-methylbutylaldehyde used for an intermediate in the production of sweetener in the present invention, N-[N-[3-(3-hydroxy-4-methoxyphenyl)-3-methylbutyl]-L-α-aspartyl]-L-phenylalanine 1-methyl ester which is important as a sweetener having a high degree of sweetness, can be easily produced efficiently on an industrial scale.

The above 3-(3-hydroxy-4-methoxyphenyl)-3-methylbutyl aldehyde can be produced easily and efficiently in a process for subjecting 3-(3-halogeno-4-methoxyphenyl)-3-methylbutyric acid, which can be obtained in the reaction of 2-halogenoanisole with 3-methylcrotonic acid, to a reaction for converting the halogen atom of a substituent group to a hydroxyl group to produce 3-(3-hydroxy-4-methoxyphenyl)-3-methylbutyric acid, followed by converting the carboxyl group of the carboxylic acid thus obtained into a formyl group.

Moreover, in the present invention, N-[N-[3-(3-hydroxy-4-methoxyphenyl)-3-methylbutyl]-L-α-aspartyl]-L-phenylalanine 1-methyl ester which is important as a sweetener having a high degree of sweetness, can be crystallized, and thus the compound can be obtained easily and conventionally in the crystalline form. Further, the present invention can be also used for a process to separate the object compound only with high purity through purification from the object compound containing impurity, and therefore, the present invention is useful industrially to an extreme.

Since a high purity of material (crystals) can be obtained and provided, the compound as such can be used directly as a sweetener component, and the compound can be easily used for a food and drink, or the like in need of a sweet taste.

The present application claims priority to JP11-288207, filed Oct. 8, 1999; JP11-288208 filed Oct. 8, 1999; JP11-294409 filed Oct. 15, 1999; and JP11-328099 filed Nov. 18, 1999, all of which are incorporated herein by reference.

1-15 (canceled).

16: A process for producing 3-(3-hydroxy-4-methoxyphenyl)-3-methylbutylaldehyde, which comprises:
converting a carboxyl group in 3-(3-hydroxy-4-methoxyphenyl)-3-methylbutyric acid to a formyl group.

17: The process as defined in claim 16, wherein said 3-(3-hydroxy-4-methoxyphenyl)-3-methylbutyric acid is produced by converting a halogen atom in 3-(3-halogeno-4-methoxyphenyl)-3-methylbutyric acid to a hydroxyl group.

18: The process as defined in claim 17, wherein said 3-(3-halogeno-4-methoxyphenyl)-3-methylbutyric acid is prepared by reacting a 2-halogenoanisole with 3-methylcrotonic acid.

19: The process as defined in claim 17, wherein said halogen atom is a chlorine atom or a bromine atom.

20: The process as defined in claim 18, wherein said reacting of a 2-halogenoanisole with 3-methylcrotonic acid comprises reacting in the presence of an acid.

21 The process as defined in claim 16, wherein said converting a carboxyl group into a formyl group comprises reducing a carboxylic acid directly to an aldehyde; or converting a carboxyl group into a hydroxymethyl group and converting said hydroxymethyl group into a formyl group.

22: A process for producing N-[N-[3-(3-hydroxy-4-methoxyphenyl)-3-methylbutyl]-L-α-aspartyl]-L-phenylalanine 1-methyl ester, which comprises:
reductively alkylating 3-(3-hydroxy-4-methoxyphenyl)-3-methylbutylaldehyde obtained by the process of claim 16 with aspartame

23: A process for producing N-[N-[3-(3-hydroxy-4-methoxyphenyl)-3-methylbutyl]-L-α-aspartyl]-L-phenylalanine 1-methyl ester, which comprises:
reductively alkylating 3-(3-hydroxy-4-methoxyphenyl)-3-methylbutylaldehyde with aspartame.

24: A compound of formula (3): 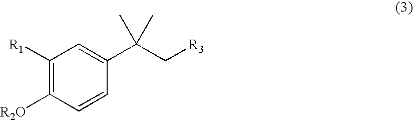
wherein R₁ is selected from the group consisting of a hydroxyl group, a halogen atom and a lower alkyloxy group having 1 to 4 carbon atoms, R₂ is a lower alkyl group having 1 to 4 carbon atoms, and R₃ is selected from the group consisting of a carboxyl group, a formyl group and a hydroxymethyl group,
provided that the compounds where R₁ is a chlorine atom or a bromine atom, and R₃ is a formyl group are excluded.

25: A compound selected from the group consisting of
3-(3-hydroxy-4-methoxyphenyl)-3-methylbutylaldehyde;
3-(3-chloro-4-methoxyphenyl)-3-methylbutyric acid;
3-(3-bromo-4-methoxyphenyl)-3-methylbutyric acid;
3-(3-hydroxy-4-methoxyphenyl)-3-methylbutyric acid; and
3-(3-hydroxy-4-methoxyphenyl)-3-methyl-1-butanol.

26: A process for producing N-[N-[3-(3-hydroxy-4-methoxyphenyl)-3-methylbutyl]-L-α-aspartyl]-L-phenylalanine 1-methyl ester, which comprises:
subjecting N-[N-[3-(3-hydroxy-4-methoxyphenyl)-3-methylbutyl]-L-α-aspartyl]-L-phenylalanine 1-methyl ester containing impurity to crystallize the compound crystallization.

27: The process as defined in claim 26, wherein said N-[N-[3-(3-hydroxy-4-methoxyphenyl)-3-methylbutyl]-L-α-aspartyl]-L-phenylalanine 1-methyl ester containing impurity is obtained by reductively akylating aspartame and 3-(3-hydroxy-4-methoxyphenyl)-3-methylbutylaldehyde or a derivative thereof.

28: The process as defined in claim 26, wherein said impurity is one or more compounds selected from the group consisting of aspartame, an aspartame derivative, a peptide derivative, an amino acid, an amino acid derivative, an aldehyde and an aldehyde derivative.

29: The process as defined in claim 26, wherein a solvent used in the crystallization is selected from the group consisting of methanol, ethanol, isopropyl alcohol, acetone, methyl ethyl ketone, methyl isobutyl ketone, methyl acetate, ethyl acetate, propyl acetate, isopropyl acetate, butyl acetate, tetrahydrofuran, acetonitrile, toluene, mixtures thereof; and mixtures thereof with water.

30: The process as defined in claim 26, further comprising removing said impurity from said N-[N-[3-(3-hydroxy-4-methoxyphenyl)-3-methylbutyl]-L-α-aspartyl]-L-phenylalanine 1-methyl ester by extracting said impurity with a solvent.

31: The process as defined in claim 30, wherein said solvent is selected from the group consisting of toluene, diethyl ether, chloroform, dichloromethane, hexane, ethyl acetate, propyl acetate, isopropyl acetate and butyl acetate.

32: A crystal of N-[N-[3-(3-hydroxy-4-methoxyphenyl)-3-methylbutyl]-L-α-aspartyl]-L-phenylalanine 1-methyl ester, which exhibits X-ray diffraction peaks at at least diffraction angles of 8.3°, 19.5° and 21.2° (2θ, CuKcαray) when examined by powder X-ray diffractometry.

33: A sweetening composition comprising the crystal of N-[N-[3-(3-hydroxy-4-methoxyphenyl)-3-methylbutyl]-L-α-aspartyl]-L-phenylalanine 1-methyl ester as defined in claim 32 and a carrier or bulking agent.

34: A food or drink comprising the crystal as defined in claim 32 as an effective ingredient.

35: A process for sweetening a food or drink, comprising
adding the crystal as defined in claim 32 to a food, a beverage, or an intermediate product used for making the food or beverage, in an amount sufficient to sweeten said food or drink.
